(19) United States
(12) Patent Application Publication
(10) Pub. No.: US 2024/0074969 A1
(43) Pub. Date: Mar. 7, 2024

(54) ORAL THIN FILMS

(71) Applicant: LTS LOHMANN THERAPIE-SYSTEME AG, Andernach (DE)

(72) Inventors: Markus MÜLLER, Troisdorf (DE); Mario FICKER, Bonn (DE); Michael LINN, Waldböckelheim (DE)

(21) Appl. No.: 18/272,205

(22) PCT Filed: Jan. 14, 2022
(86) PCT No.: PCT/EP2022/050790

§ 371 Date: Jul. 13, 2023

(30) Foreign Application Priority Data
Jan. 15, 2021 (DE) 10 2021 100 718.6

(51) Int. Cl. A61K 9/00; A61K 47/34

(52) U.S. Cl. CPC A61K 9/0056; A61K 47/34

(57) ABSTRACT

The present invention relates to a method for producing a multi-layer oral thin film comprising the steps of a) providing a solution, an emulsion, a foam or a suspension comprising at least one first polymer, b) processing the solution, the emulsion, the foam or the suspension from step a) in order to obtain a polymer film, c) applying a powder comprising at least one second substance, which preferably differs from the first polymer, to the polymer film, and d) forming a composite between the polymer film and the powder applied in step c), to a multi-layer oral thin film obtainable by this method, and to the use of a multi-layer oral thin film obtainable by this method as a medicament.

The present invention relates to a method for producing a multi-layer oral thin film, an oral thin film obtainable by this method, and use thereof as a medicament.

Oral thin films (OTFs) are thin films generally containing at least one pharmaceutically active agent that are placed directly in the oral cavity or against the oral mucosa and dissolve there. These oral thin films can be constructed as single- or multi-layer systems. The pharmaceutically active agent may be dissolved, emulsified or dispersed in the film. Oral thin films, however, are not just limited to the administration of medicaments, but can also be used in the food sector and also in dental care or as mouth fresheners.

In various cases, it may be necessary for an OTF to have a long residence time in the patient's mouth. However, due to permanent saliva flow and movements in the mouth, the films are subject to permanent erosion. To prevent this, the OTF can be provided with one or more slowly soluble or insoluble backing layer(s).

However, it might also be necessary to apply rapidly soluble substances, such as flavourings, buffer substances, salts or sweeteners, as backing layer(s) to the OTF.

In the case of multi-layer OTFs known from the prior art, these backing layers are applied by means of a double or multiple coating or are glued on by means of an adhesive layer.

A disadvantage of these known methods is that the backing layer has to be produced separately and then bonded to the actual film by double coating or glued on via an adhesive layer. Double or multiple coating leads to increased thermal stress, especially for the pharmaceutically active agent contained, and also often leads to poorly adhering composites. Due to the diffusion of the solvent into the lower film during double or multiple coating, the solvent often has to be dried out again at great expense.

For the bonding of the different layers, it is also necessary to produce the backing layer separately and then bond it. Especially for backing layers that are to dissolve slowly during application, polymers must be chosen that, in addition to their poorer solubility, also have a high viscosity in the polymer solution. This leads to low solids contents and thus to long drying times. Furthermore, the films must be very thin so that they are not perceived as disturbing, which makes them filigree and difficult to handle.

The aim of the present invention lies in overcoming the above-mentioned disadvantages of the prior art. Especially, the aim of the present invention is to provide a method for producing a multi-layer oral thin film in which the individual layers do not have to be produced separately and do not have to be joined together to form a composite by a lamination of individual separately dried layers. It is further intended to provide a multi-layer oral thin film, wherein the individual layers are preferably also not bonded together by a separate adhesive layer. In addition, thermal stress on the pharmaceutically active agent contained should be avoided as far as possible.

The above aim is addressed by a method for producing a multi-layer, preferably two-layer, oral thin film according to claim 1, i.e. by a method for producing a multi-layer oral thin film comprising the steps of
a) providing a solution, an emulsion, a foam or a suspension comprising at least one first polymer,
b) processing the solution, the emulsion, the foam or the suspension from step a) in order to obtain a polymer film,
c) applying a powder comprising at least one second substance, which preferably differs from the first polymer, to the polymer film, and
d) forming a composite between the polymer film and the powder applied in step c).

Preferably, the powder comprising at least one second polymer, which preferably differs from the first, is applied to the polymer film in step c) before the latter has completely dried.

The powder applied in step c) preferably comprises a second polymer, wherein the second polymer preferably differs from the first polymer.

The polymer applied in step c) may also comprise non-polymeric substances, such as sweeteners, flavourings, buffer substances, salts or even pharmaceutically active agents.

An advantage of this method is that no separate production of different layers is necessary here. Only a first polymer film is produced, onto which a second substance, preferably a second polymer, is applied, preferably before the first polymer film has completely dried.

Such an incompletely dried polymer film is preferably distinguished in that it has a certain tackiness, which causes the second substance, preferably the second polymer, to adhere to the polymer film and form a composite. By subsequently drying the composite, a multi-layer oral thin film can thus be provided. Thus, a multi-layered oral thin film can be provided, wherein the individual layers do not have to be produced separately and joined together by bonding or lamination. In addition, the multi-layer oral thin film is not excessively thermally stressed.

A polymer film that is not completely dried is understood to be a polymer film that has a residual solvent content of from 0.1 to 10 wt. %.

If the residual solvent content in the film is too low, the film can be additionally sprayed with solvent to achieve sufficient moisture on the surface of the film.

Preferably, however, the polymer film is not further treated prior to the application of the second substance, especially not by the application of another substance that promotes the adhesion of the second substance, such as an adhesive.

The solution, the emulsion, the foam or the suspension is processed by the usual methods, such as spreading, extrusion or printing.

The method according to the invention is further preferably distinguished in that the forming of the composite comprises a drying of the composite by heating the two-layer composite to a temperature above the melting point of the first and/or the second substance, preferably the second polymer.

The composite does not necessarily have to be dried (in the sense of removing solvent). It may also be sufficient to heat or melt the sprinkled-on second substance, preferably the second polymer, or to reach the glass transition temperature. It is also conceivable to press on the sprinkled-on second substance, preferably the second polymer.

This has the advantage that at least one of the at least two different substances or polymers softens or melts, thus resulting in a more stable composite formed of the layers.

In an alternative embodiment of the method according to the invention, it may also be sufficient to heat the composite to a temperature below the melting point of the polymers used in order to already achieve stabilisation of the composite. Suitable temperatures are, for example, from about 50° C. to about 120° C., preferably to about 100° C., especially preferably to about 80° C. This is preferred if the melting point of the first or second substance, preferably of the polymer, is so high that heating to a temperature above these melting points impairs the further ingredients of the multi-layer oral thin film, especially any pharmaceutically active substance present.

The method according to the invention is further preferably distinguished in that the polymer film has a residual moisture of about 0.1 to 10 wt. % at the time of application of the second substance, preferably the at least one second polymer. A residual moisture is understood to mean the content of solvent in wt. %.

If there is less residual moisture in the polymer film, then the second substance, preferably the at least one second polymer, which differs from the first, no longer adheres well enough to the polymer film.

It is also preferred that the polymer film has a certain tackiness. A film is tacky if it can act as an adhesive or pressure sensitive adhesive as defined in DIN EN 923:2016-03. This helps the at least one second polymer, which differs from the first, to adhere to the surface of the polymer film.

The method according to the invention is also preferably distinguished in that the at least one second polymer is applied as powder. The powder is preferably dry, i.e. it has a residual moisture, as defined above, of less than 10 wt. %.

Preferably, this powder, which is preferably a dry powder, has a mean particle size of less than 500 μm, preferably of less than 200 μm, determined by dynamic light scattering.

The method according to the invention is further distinguished in that the at least one first polymer comprises a water-soluble and/or water-swellable polymer.

Water-soluble polymers comprise chemically very different natural or synthetic polymers, the common feature of which is their solubility in water or aqueous media. A precondition for this is that these polymers have a number of hydrophilic groups sufficient for the water solubility and are not crosslinked. The hydrophilic groups may be non-ionic, anionic, cationic and/or zwitterionic.

Water-swellable polymers are polymers that increase their volume through the penetration of water.

The method according to the invention is further distinguished in that the at least one first polymer is selected from starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums.

These polymers have the advantage that they are compatible with a wide range of pharmaceutically active agents and are also largely harmless to a patient for whose treatment the multi-layer oral thin film according to the invention is used.

These polymers additionally have the advantage that they form a thin stable film when dried, which dissolves upon application to the mucosa or in the oral cavity and thus releases the active agent. This has the advantage of a quick availability of the active agent as well as a residue-free administration of the active agent.

The method according to the invention is additionally preferably characterised in that the at least one second polymer comprises a water-insoluble polymer.

The method according to the invention may additionally preferably be characterised in that the at least one second polymer comprises a slowly water-soluble polymer or a water-soluble polymer.

Water-soluble polymers comprise chemically very different natural or synthetic polymers, the common feature of which is their solubility in water or aqueous media. A precondition is that these polymers have a number of hydrophilic groups sufficient for the water solubility and are not crosslinked. The hydrophilic groups may be non-ionic, anionic, cationic and/or zwitterionic.

Water-soluble polymers preferably have a solubility in water of greater than 100 g/L at 25° C.

Preferably, the at least one second polymer comprises polyethylene oxide, preferably having an average molecular weight of about 7,000,000 g/mol.

The use of such a water-insoluble or poorly water-soluble polymer has the advantage that a layer is formed on one side of the multi-layer oral thin film and protects the multi-layer oral thin film from erosion or rapid dissolution.

The method according to the invention is additionally preferably characterised in that the polymer film, in addition to the at least one first polymer, additionally contains at least one plasticiser.

Plasticisers are liquid or solid, indifferent organic substances, preferably with low vapour pressure, which can physically interact with highly polymeric substances without chemical reaction, preferably through their dissolving and swelling properties, but possibly also without such properties, and can form a homogeneous system with them. Plasticisers impart certain desired physical properties to the structures or coatings produced with them, such as reduced freezing temperature, reduced glass transition temperature, increased ability to change shape, increased elastic properties, reduced hardness and possibly increased adhesion. They belong to the group of plastic additives.

It is preferred that the at least one plasticiser comprises glycerol, triacetin, polyethylene glycol, especially polyethylene glycol 200, sorbitol, water, ethanol and/or tributyl citrate.

This is especially advantageous as many water-soluble polymers can be made tacky by the addition of plasticisers.

If a plasticiser is present in the polymer film, it is preferred that the amount of the at least one first polymer in the polymer film is about 25 to 90 wt. %, preferably about 35 to 85 wt. %, especially preferably about 40 to 80 wt. %, in relation to the total weight of the polymer film.

If a plasticiser is present in the polymer film, it is preferred that the amount of the at least one plasticiser in the polymer film is about 0.1 to 50 wt. %, preferably about 0.5 to 25 wt. %, in relation to the total weight of the matrix layer.

If too little or too much plasticiser is used, the mixture for example is either not tacky or a workable compound cannot be provided from the outset.

The use of a polymer film made tacky by the addition of at least one plasticiser has the advantage that the second substance, preferably the second polymer, applied to the polymer film adheres especially well.

Especially preferred is a mixture of polyvinyl alcohol and/or polyvinyl pyrrolidone as the first water-soluble polymer and glycerol as the plasticiser, preferably in a ratio of about 80:20 wt. %.

The method according to the invention is additionally preferably characterised in that the polymer film comprises at least one pharmaceutically active agent, and/or wherein at least one pharmaceutically active agent is applied to the polymer film either alone or together with the at least one second polymer.

If the at least one pharmaceutically active agent is contained in the polymer film, the at least one pharmaceutically active agent is preferably present dissolved, emulsified or suspended therein.

If the at least one pharmaceutically active agent is applied to the polymer film alone or together with the at least one second polymer, it is preferably applied in powder form. The at least one pharmaceutically active agent is preferably present as a dry powder, i.e. it has a residual moisture content, as defined above, of less than 10 wt. %.

Preferably, the powdered at least one pharmaceutically active agent has a mean particle size of less than 500 μm, preferably less than 200 μm, measured by dynamic light scattering.

The at least one pharmaceutically active agent is not limited, but includes all pharmaceutically active agents that are administrable by means of an OTF.

Suitable pharmaceutically active agents are preferably selected from the group consisting of analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active agents, antibiotics, chemotherapeutics and narcotics.

In a further alternative embodiment of the multi-layer oral thin film, the at least one pharmaceutically active agent is applied as a second substance to the polymer film.

Alternatively, a buffer substance, a sweetener and/or a salt is applied.

The method according to the invention is preferably characterised in that at least one further layer, preferably a further polymer layer, is applied to the side of the two-layer composite on which the second substance, preferably the second polymer, has not been applied.

This can be done by conventional methods known to a person skilled in the art, such as lamination and/or bonding. This further at least one layer may comprise the same and further pharmaceutically active agents or buffer substances, sweeteners, flavourings or salts. Furthermore, special mucoadhesive polymers may be used in this layer to increase the adhesion of the multi-layer oral thin film to the patient's mucosa.

The method according to the invention is preferably characterised in that the polymer film additionally contains at least one auxiliary substance selected from the group comprising colouring agents, flavourings, sweeteners, taste-masking agents, surfactants, enhancers, pH regulators, preservatives and/or antioxidants, and/or at least one of these auxiliary substances is applied together with the at least one second polymer to the polymer film.

If the additional at least one auxiliary substance is applied to the polymer film together with the second substance, preferably the second polymer, it is preferably applied in powder form. The at least one auxiliary substance is preferably present as a dry powder, i.e. it has a residual moisture content, as defined above, of less than 10 wt. %.

It is also possible to apply to the polymer film a superabsorber as a second substance, alone or together with the at least one second substance. This also allows the application of liquids, which are absorbed by the superabsorber.

The method according to the invention is additionally preferably characterised in that the multi-layer oral thin film has an area density of from 50 to 400 g/m 2.

The present invention further relates to a multi-layer oral thin film obtainable by the method described above.

The present invention furthermore relates to a multi-layer oral thin film obtainable by the method described above for use as a medicament.

The invention will be explained below by means of non-limiting examples.

### EXAMPLES

### Example 1

A polymer film according to formulation 1 was applied to a siliconised coating carrier. The polymer used, polyvinylpyrrolidone, has the property that it is also tacky when provided with the plasticiser glycerol. Immediately after spreading, the surface was sprinkled with finely sieved (<160 μm) polyethylene oxide (Polyox VVSR-303; molecular weight 7,000,000). The excess powdery polymer was brushed off with a brush and the film was then dried. Lastly, the film was heated for 5 min to a temperature above the melting point of both polymers. The resulting composite could be easily removed from the siliconised coating substrate and further processed. If desired, the composite can be rolled onto another laminate. The result is a composite that has a mucoadhesive layer on one side and a layer that protects the film from erosion on the other side.

TABLE 1
with formulation 1:

| Material | Proportion [wt. % dry] | Description |
|---|---|---|
| PVP VA64 | 80 | Polymer |
| Glycerol | 20 | Plasticiser |

### Example 2

Three different categories are presented below to illustrate the invention.
1. Examples in which Kollicoat MAE 100 is sprinkled as a powder directly onto a compound and then dried.
2. Examples with adhesive layer, wherein an adhesive layer was produced and laminated onto a foam laminate provided in advance. Subsequently, the powdered materials were sprinkled on, covered, and pressed on with a roller.
3. Examples with polyox, wherein a polyox laminate was produced, powder applied and then heated above the melting point of polyox.

### Category 1:

### Production of the Base Film+Powder Application:

The compound according to the following Table 2 was produced and the powdered materials were immediately sprinkled on. Subsequently, drying was carried out in a drying cabinet.

TABLE 2

| Composition: | | | |
|---|---|---|---|
| Starting | | Proportion | Proportion |
| material | Function | mg/m² | % |
| Kollicoat | Polymer | 9.742 | 20.95 |
| MAE100-55 | | | |
| Kollicoat MAE | Polymer | 29.272 | 62.95 |
| 100 P | | | |
| Triethyl citrate | Plasticiser | 4.650 | 10 |
| Cherry US | Flavouring | 1.395 | 3 |
| Saccharin NA | Sweetener | 0.930 | 2 |
| Sucralose | Sweetener | 0.465 | 1 |
| Menthol | Flavouring | 0.047 | 0.1 |
| | Total | 46.5 | 100 |

The weight was determined using 10 cm 2 test specimen.
Coating thickness: 150 μm
Coating carrier: 75 μm PET AB1 on AB
Drying: 15 min at 70° C.
Area density: 46.5 cm/m²

9 different powdered materials were sprinkled on according to the following Table 3.

TABLE 3

| | | | | Total | Powder |
|---|---|---|---|---|---|
| | | Powder | | weight | weight |
| Base film | Technique | Function | Material | [g/m²] | [g/m²] |
| Kollicoat | Sprinkled | Superabsorber | Kelkogel | 75.8 | 29.30 |
| MAE | on | Ion | Amberlite | 75.8 | 29.30 |
| 100 Film | before | exchanger | IRP64 | | |
| Area | drying | Flavouring | Firmenich | 93.7 | 47.20 |
| density | | | Peach | | |
| 46.5 | | Masking | Mane Bitter | 98.1 | 51.60 |
| [g/m²] | | | Masking | | |
| | | Buffer | Trisodium | 417.1 | 370.60 |
| | | | citrate | | |
| | | Sweetener | Sucralose | 66.9 | 20.40 |
| | | Polymer | Kollidon VA | 108.8 | 62.30 |
| | | (mucoadhesive) | 64 | | |
| | | API | Caffeine | 77 | 30.50 |
| | | API mixture | Mixture for | 143.4 | 96.90 |
| | | | melting | | |

### Category 2:

### Production of an Adhesive Laminate+Powder Application:

An adhesive layer was produced and laminated onto a foam laminate provided in advance. The composition of the adhesive layer and of the foam laminate are summarised in Table 4. Subsequently, the powdered materials were sprinkled on, covered, and pressed on with a roller. The samples were taken similarly as for Category 1.

TABLE 4

| | Composition | | | |
|---|---|---|---|---|
| | Starting | | Proportion | Proportion |
| | material | Function | mg/m² | % |
| Foam | PVA 4-88 | Polymer | 77.60 | 52.33 |
| laminate* | | | | |
| 100 g/m² | FD&C red no. 40 | Colouring | 0.40 | 0.27 |
| | | agent | | |
| | Saccharin NA | Sweetener | 2.00 | 1.35 |
| | Sucralose | Sweetener | 4.00 | 2.70 |
| | Glycerol | Plasticiser | 9.00 | 6.07 |
| | Cherry US | Flavouring | 6.00 | 4.05 |
| | Menthol | Flavouring | 1.00 | 0.67 |
| Adhesive | Vinyl alcohol | Polymer | 38.64 | 26.06 |
| layer: | (VA64) | | | |
| 48.3 g/m² | Glycerol | Plasticiser | 9.66 | 6.51 |
| | | Total | 148.3 | 100.00 |

*Foamed with ambient air; alternatively, foaming with nitrogen is possible.

Coating thickness: 150 μm
Coating carrier: 75 μm PET AB1 on AB
Drying: 10 min at RT and 8 min at 70° C.
Area density: 148.3 cm/m 2

8 different powdered materials were sprinkled on according to the following Table 5.

TABLE 5

| | | | | Total | Powder |
|---|---|---|---|---|---|
| Base | | Powder | | weight | weight |
| film | Technique | Function | Material | [g/m²] | [g/m²] |
| Foam + | Pressed | Superabsorber | Kelkogel | 161.9 | 13.60 |
| Adhesive | onto | Ion exchanger | Amberlite | 155.1 | 6.80 |
| layer | adhesive | | IRP64 | | |
| Area | layer | Flavouring | Firmenich | 159.1 | 10.80 |
| density | | | Peach | | |
| 148.3 g/ | | Masking | Mane Bitter | 173.4 | 25.10 |
| m² | | | Masking | | |
| | | Buffer | Trisodium | 403.2 | 254.90 |
| | | | citrate | | |
| | | Polymer | Kollidon VA | 164.1 | 15.80 |
| | | (mucoadhesive) | 64 | | |
| | | API | Caffeine | 149.1 | 0.80 |
| | | API mixture | Mixture for | 154 | 5.70 |
| | | | melting | | |

### Category 3:

### Production of a Polyox Laminate+Powder Application:

The compound according to Table 6 was coated and dried. After drying, the powdered materials were sprinkled on, and the products were covered, placed on a tray and weighted down with another tray from above. Afterwards, the products were placed between the trays in the drying cabinet and melted at 120° C. After cooling, the samples were taken similarly as for Category 1.

TABLE 6

| Composition | | | |
|---|---|---|---|
| Starting | | Proportion | Proportion |
| material | Function | mg/m² | % |
| Polyox | Polymer | 92.63 | 97.5 |
| Glycerol | Plasticiser | 2.19 | 2.3 |
| FD&C red | Colouring | 0.19 | 0.2 |
| no. 40 | agent | | |
| | Total | 95.00 | 100 |

Coating thickness: 300 μm
Coating carrier: 100 μm PET TSP
Drying: 15 min at 70° C.
Area density: 95 cm/m 2

### Melting Step:

Cover film: 75 μm PET AB1 on AB
Melting: 10 min at 120° C.

8 different powdered materials were sprinkled on according to the following Table 7.

TABLE 7

| | | | | Total | Powder |
|---|---|---|---|---|---|
| Base | | Powder | | weight | weight |
| film | Technique | Function | Material | [g/m²] | [g/m²] |
| Polyox | Melted | Superabsorber | Kelkogel | 119.8 | 73.30 |
| Area | | Ion exchanger | Amberlite IRP64 | 94.2 | 47.70 |
| density | | Flavouring | Firmenich Peach | 168.4 | 121.90 |
| 95 g/m² | | Masking | Mane Bitter | 146 | 99.50 |
| | | | Masking | | |
| | | Buffer | Trisodium citrate | 482.3 | 435.80 |
| | | Polymer | Kollidon VA 64 | 159.9 | 113.40 |
| | | (mucoadhesive) | | | |
| | | API | Caffeine | 196.7 | 150.20 |
| | | API mixture | Mixture for | 96.6 | 50.10 |
| | | | melting | | |

1. A method for producing a multi-layer oral thin film comprising the steps of
a) providing a solution, an emulsion, a foam or a suspension comprising at least one first polymer,
b) processing the solution, the emulsion, the foam or the suspension from step a) in order to obtain a polymer film,
c) applying a powder comprising at least one second substance, which preferably differs from the first polymer, to the polymer film, and
d) forming a composite between the polymer film and the powder applied in step c).

2. The method according to claim 1, wherein the forming of the composite comprises the heating of the composite to a temperature above the melting point of the first and/or the second substance.

3. The method according to claim 1, wherein the polymer film has a residual moisture of from 0.1% to 10 wt. % at the time of application of the at least one second substance.

4. The method according to claim 1, wherein the at least one second substance comprises at least one second polymer, which differs from the first.

5. The method according to claim 1, wherein the at least one first polymer comprises a water-soluble or water-swellable polymer.

6. The method according to claim 1, wherein the at least one first polymer is selected from starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and/or natural gums.

7. The method according to claim 1, wherein the at least one second substance comprises a water-insoluble polymer.

8. The method according to claim 1, wherein the at least one second polymer comprises polyethylene oxide.

9. The method according to claim 1, wherein the polymer film additionally contains at least one plasticiser.

10. The method according to claim 1, wherein the polymer film comprises at least one pharmaceutically active agent, and/or wherein at least one pharmaceutically agent is applied as second substance to the polymer film, wherein the pharmaceutically active agent is selected from the group consisting of analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active agents, antibiotics, chemotherapeutics and narcotics.

11. The method according to claim 1, further comprising the application of at least one further layer to the side of the composite to which the second polymer has not been applied.

12. The method according to claim 1, wherein the polymer film additionally contains at least one auxiliary substance selected from the group comprising colouring agents, flavourings, sweeteners, taste-masking agents, surfactants, enhancers, pH regulators, preservatives and/or antioxidants, and/or at least one of these auxiliary substances is applied together with the at least one second polymer to the polymer film.

13. The method according to claim 1, wherein the multi-layer oral thin film has an area density of from 50 to 400 g/m 2.

14. A multi-layer oral thin film obtained by the method according to claim 1.

15. (canceled)

16. A method of administrating a medicament comprising providing a multilayer oral thin film produced according to the method of claim 1 to a subject.

17. The method of claim 1 wherein the second substance differs from the first polymer.

18. The method according to claim 4, wherein the second polymer comprises a dry powder.

19. The method according to claim 18, wherein the dry powder has a particle size of less than 500 μm.

20. The method according to claim 8, wherein the polyethylene oxide has an average molecular weight of 7,000,000 g/mol.

21. The method according to claim 11, wherein the further layer comprises a polymer layer.
